(19) United States
(12) Patent Application Publication
(10) Pub. No.: US 2018/0014724 A1
(43) Pub. Date: Jan. 18, 2018

(54) Method and System for Analysis of Diagnostic Parameters and Disease Progression

(71) Applicant: Dariusz Wroblewski, San Diego, CA (US)

(72) Inventor: Dariusz Wroblewski, San Diego, CA (US)

(21) Appl. No.: 14/999,951

(22) Filed: Jul. 18, 2016

(51) Int. Cl. A61B 3/00; A61B 3/024; A61B 5/00; G06F 19/00; A61B 3/02

(52) U.S. Cl. CPC A61B 3/0025; G06F 19/345; G06F 19/3431; G06F 19/322; A61B 3/022; A61B 5/7264; A61B 5/4842; A61B 3/024; A61B 5/7275

(57) ABSTRACT

A system and method of identifying a change in an eye to detect a probability of disease progression is provided. A database of eye examination maps of different eyes is employed, with each eye examination map including visual field information. Maps of eyes from the database are then selected that show no change between their examinations. Measurement data from the selected eye maps is then generated by collecting measurements made at visual field locations from each selected eye map. Simulated eye examination maps are then generated by randomly sampling a value from the measurement data. A global index is then employed to generate a population of possible global index values for the simulated eye examination maps. The simulated eye examination maps and the corresponding global index values are used to provide a measure of the probability of disease progression.

### FIELD OF THE INVENTION

The present invention generally relates to methods of diagnosing disease progression. More particularly, the invention concerns a method and apparatus to diagnose disease progression with a limited number of diagnostic measurements per patient.

### BACKGROUND OF THE INVENTION

Glaucoma is a progressive optic neuropathy that causes an irreversible, but often preventable, loss of vision. It is the leading cause of blindness worldwide and is particularly common in the elderly. Primary open-angle glaucoma (POAG) is the prevailing form in the United States and leads to characteristic changes in structure and visual function. Detection of early glaucomatous damage and accurate, quantitative assessment of its progression are the most important challenges in management of the disease, and have an immense impact on the treatment outcome and the patients' quality of life. Treatment efficacy depends on the ability to differentiate stable from progressive disease.

Generally, structural and functional testing is used in the determination of glaucoma progression, but in clinical practice the progression detection is largely dependent on functional testing performed with standard automated perimetry (SAP) devices. Perimetry refers to the systematic measurement of the visual field, or retinal sensitivity as a function of spatial location, and is an essential component of defining the extent and progression of glaucoma. Currently, standard automated perimetry or white on white perimetry is the most common form of visual field testing. Here, a white stimulus is projected on a white background to determine threshold values of retinal sensitivity

Using a SAP device, a health practitioner conducts an examination of a patient's eyes and the SAP device produces a test output in the form of an individual patient's data. However, the practitioner must compare each test output with preceding and succeeding tests to determine any emerging patterns over the course of numerous tests. However, it is still difficult for the practitioner to easily and quickly detect the progress of disease without time consuming, careful analysis.

Comparing a series of successive tests taken over a period of time for the same patient enables the practitioner to determine the progress of a disease. However, mistakes can be made during analysis of the test data, and the requirement for multiple tests makes the determination of glaucoma progression a problematic issue.

Therefore, there remains a need to overcome one or more of the limitations in the above-described, existing art. The discussion of the background to the invention included herein is included to explain the context of the invention. This is not to be taken as an admission that any of the material referred to was published, known or part of the common general knowledge as at the priority date of the claims.

### BRIEF DESCRIPTION OF THE DRAWINGS

FIG. 1 shows the output of a visual field test in the form of a retinal sensitivity map of a patient known to have glaucoma and shows loss of peripheral vision in the upper hemifield;

FIG. 2 is a block diagram illustrating elements of one embodiment of the present invention;

FIG. 3 is a block diagram illustrating elements of one embodiment of the present invention;

FIG. 4 is a second block diagram illustrating elements of one embodiment of the present invention;

FIGS. 5a-b are graphical outputs generated by one embodiment of the present invention;

FIG. 6 is a graphical output generated by another embodiment of the present invention;

FIG. 7 is a map generated by an embodiment of the present invention; and

FIG. 8 is a graphical output generated by yet another embodiment of the present invention.

It will be recognized that some or all of the Figures are schematic representations for purposes of illustration and do not necessarily depict the actual relative sizes or locations of the elements shown. The Figures are provided for the purpose of illustrating one or more embodiments of the invention with the explicit understanding that they will not be used to limit the scope or the meaning of the claims.

### DETAILED DESCRIPTION OF THE PREFERRED EMBODIMENTS

In the following description, for the purposes of explanation, numerous specific details arc set forth in order to provide a thorough understanding of the visual field progression system (VFPS) that embodies principals of the present invention. It will be apparent, however, to one skilled in the art that the VFPS may be practiced without some of these specific details. Throughout this description, the embodiments and examples shown should be considered as exemplars, rather than as limitations on the VFPS. That is, the following description provides examples, and the accompanying drawings show various examples for the purposes of illustration. However, these examples should not be construed in a limiting sense as they are merely intended to provide examples of the VFPS rather than to provide an exhaustive list of all possible implementations of the VFPS.

Unless defined otherwise, all technical and scientific terms used herein have the same meaning as is commonly understood by one of skill in the art to which this invention belongs. In event the definition in this section is not consistent with definitions elsewhere, the definitions set forth in this section will control.

Specific embodiments of the invention will now be further described by the following, non-limiting examples which will serve to illustrate various features. The examples are intended merely to facilitate an understanding of ways in which the invention may be practiced and to further enable those of skill in the art to practice the invention. Accordingly, the examples should not be construed as limiting the scope of the invention. In addition, reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment. Furthermore, the particular features, structures or characteristics may be combined in any suitable manner in one or more embodiments. As used herein, the “present invention” refers to any one of the embodiments of the invention described herein, and any equivalents. Furthermore, reference to various feature(s) of the “present invention” throughout this document does not mean that all claimed embodiments or methods must include the referenced feature(s).

Early detection of glaucoma is critical. However, detecting the minor changes to the visual field (i.e., what we see) caused by the early stages of glaucoma is extremely difficult to detect. This is because an eye test, or a visual field test, is an intrinsically “noisy” test. Patient performance during eye testing can be affected by stress, fatigue, and other factors. Also, normal vision defects (blind spots, “floaters,” and age related visual deterioration) can affect test results. All of these factors contribute to “visual noise,” or “measurement noise” which is the unwanted errors associated with eye testing.

To minimize the effects of visual noise, many qualitative and quantitative statistical methods have been developed. However, there still exists a need for a system and method that can reliably detect the early onset of glaucoma, its progression, and also detect or evaluate other diseases by differentiating between visual noise or other types of noise and disease. The difficulties in assessing the visual field progression are in a large extent due to the fact that perimetry is an intrinsically noisy test, especially in the region of lower sensitivities. Changes in the visual field may be associated with disease progression but in order to identify the changes that are statistically significant one needs a realistic model of the visual or measurement noise. Quantification of visual noise in perimetric measurement is complicated by the fact that only a limited number of exams are performed for a given eye. Thus, the standard approach, in which one would evaluate variation in a large number of replicate measurements, is not feasible.

The present invention, presents a self-consistent system and method that produces a comprehensive model of the measurement noise. It also enables quantitative assessment of the probability of change in a global index (progression/regression) and, by extension, disease progression. In contrast to other approaches, a statistically significant change in the global index value (that is, a change that cannot be attributed to intrinsic, random fluctuations and thus, is mostly due to the change in patient condition) can be determined by comparison of only two diagnostic results. Acquisition and analysis of a larger number of diagnostic results (time histories) helps in progression determination but is not necessary.

Structural and functional testing is used in the determination of glaucoma progression but in clinical practice the progression detection is largely dependent on functional testing performed with standard automated perimetry (SAP) devices. For example, the Humphrey Visual Field Analyzer is a SAP device, and it projects a series of white light stimuli of varying intensities (brightness), throughout a uniformly illuminated bowl. The patient presses a button to indicate when he/she sees the stimulus. This assesses the retina's ability to detect a stimulus at specific points within the visual field. This is called retinal sensitivity and is recorded in ‘decibels’ (dB). Every log order change in light intensity corresponds to 10 dB, such that the machine can measure sensitivities over a 50 dB range. Test locations unable to see a stimulus of 10,000 apostilb (asb) are assumed to be totally blind (though they may still see brighter lights (or other visual stimuli such as motion), and are assigned a value of 0 dB. The threshold values reported for each location reflect the extent to which light can be dimmed and still detected. For example, a value of 30 dB indicates that the stimulus can be dimmed 1000-fold to 10 asb.

The normal eye can detect stimuli over a 120° range vertically and a nearly 160 degree range horizontally. From the point of fixation, stimuli can typically be detected 60° superiorly, 70° inferiorly, 60° nasally, and 100 degrees temporally, though the true extent of the visual field depends on several features of the stimulus (size, brightness, motion) as well as the background conditions. The field of vision is often depicted as a three dimensional hill, with the peak sensitivity to stimuli occurring at the point of fixation under photopic conditions, decreasing rapidly in the 10° around fixation, and then decreasing very gradually for locations further in the periphery. Nerve fibers pass through the sclera at the optic nerve head, typically 10-15° nasal to fixation. At this location, no photoreceptors are present, creating a “blind spot.”

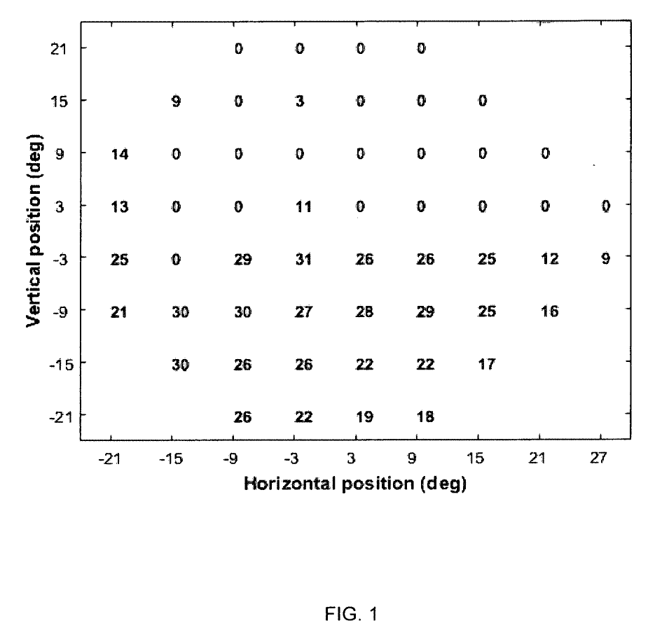

Referring now to FIG. 1, an example of an eye retinal sensitivity map obtained with a standard automatic perimeter such as a Humphrey Visual Field Analyzer is illustrated. Numbers indicate sensitivity in dB units. This example is for a glaucomatous eye and shows loss of peripheral vision in the upper hemifield. Retinal sensitivity maps represent raw values of a patient's retinal sensitivity at specific retinal points in dB. Higher numbers equate to higher retinal sensitivities. Sensitivity is greatest in the central field and decreases towards the periphery. Normal values are approximately 30 dB while recorded values of <0 dB equate to no sensitivity measured.

Most glaucoma progression studies have concentrated on analysis of visual field progression and a large variety of qualitative and quantitative statistical methods have been proposed. Quantitative interpretation of visual field testing is facilitated by the use of visual field indices, or global indices, which are based on comparison with age-corrected fields for normal individuals and include, among others, the mean deviation (MD), pattern standard deviation (PSD), and the visual field index (VFI). Several global indices are discussed below.

The average of deviations across all test locations is referred to as the mean deviation (MD). Subjects, who are able to see dimmer stimuli than others of similar age will have positive values for their MD, while subjects who require brighter stimuli will have negative MD values. MD values for reliable tests typically range from +2 dB to −30 dB. Total Deviation takes the raw data results for each test point of an exam and compares the results against an established age-corrected normal. The deviation is the difference between what is “statistically” normal for a particular test point and the measured value at this test point. If the patient saw better than normal, the result will be a positive deviation, if the patient saw worse, then the deviation will be negative. From these deviations a probability is determined which indicates whether the deviations are non-significant or if significant, how much (is this deviation present in <5% of the population?, <2% of the population?, etc.). Pattern Deviation is, in simple terms, an offset--up or down—in the Total Deviation. The amount of offset is called the elevator. This shifting of the Total Deviation field filters out noise caused by such things as cataracts, small pupils, or “supernormal” vision making the results more sensitive to localized scotomas. As with Total Deviation, from these pattern deviations a probability can be determined indicating how significant this deviation is.

Visual field loss in glaucoma is frequently non-uniform, and thus a measure which quantifies irregularities is desirable. Pattern standard deviation (PSD) measures irregularity by summing the absolute value of the difference between the threshold value for each point and the average visual field sensitivity at each point (equal to the normal value for each point+the MD). Visual fields with the age-normal sensitivity at each point will have a PSD of 0, as will visual fields in which each point is uniformly depressed from the age-normal value. Thus, the largest PSD will be registered for focal, deep visual field defects. Near-normal and severely damaged visual fields will both have low PSD.

The visual field index (VFI) is a weighted mean of a percentage of the sensitivities expected in a healthy patient of the same age.

Despite considerable research in this area, there is still no quantitative clinical standard for evaluation of glaucoma progression and the detection of visual field (VF) change, as an indicator of glaucoma progression, remains a difficult task. Studies comparing different approaches have revealed large differences in their ability to detect VF progression.

The present invention is a novel, empirical, non-parametric approach to evaluation of visual field data, which uses resampling of eye sensitivity data to obtain estimates of confidence intervals (or, statistical variation) in evaluation of global indices (such as MD, PSD, VFT and others). In principle, any global index can be used for progression evaluation and performance of different indices may be compared by evaluating the model self-consistency—the agreement between test specificity and assumed confidence intervals. A visual noise model for eye sensitivity measurements is derived from visual field data for a large number of normal and glaucomatous eyes that are considered stable—i.e., eyes that should produce identical visual fields in the absence of visual, or measurement noise. Then, for a given series of perimetry exams, the invention assesses if observed changes in a global index are due to statistical variation (i.e., measurement noise) or, to actual changes in the visual field.

As opposed to conventional methods, only two consecutive exams are required to calculate the probability of disease progression. The present invention does not depend on the clinical “ground truth” (clinical progression finding, which is not well defined for glaucoma) but on the properties gleaned from the perimetry data.

In a preferred embodiment, the present invention uses population studies (data collected from many patients) with a limited number of replicate measurements for an individual patient, to empirically determine measurement, or visual noise for medical diagnostic instruments and biomedical instruments that simultaneously measure large number of features but typically produce small number of replicate measurements. As an example, below, the present invention will be applied to the visual field test and its application to the determination of glaucoma progression.

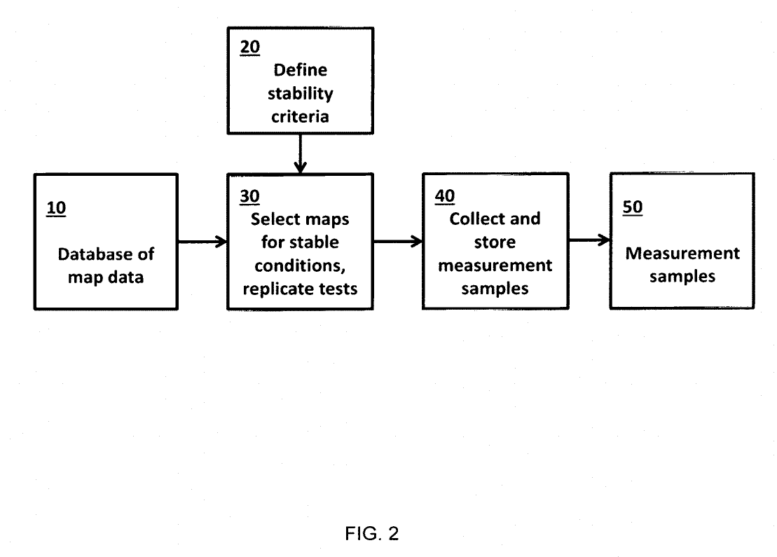

Referring now to FIG. 2, the process of generating measurement samples of patient data is illustrated. At 10, a database of eye exams in the form of retinal sensitivity maps, as shown in FIG. 1, is accessed. The database comprises replicate exams (repeat exams taken on the same date or close date) and multiple exams for stable (non-disease-progressing) eyes.

At 20, stability criteria are determined. Definition of a stable eye is somewhat arbitrary but helpful for assessment of long-term fluctuations in the visual field exam (as opposed to short-term fluctuations that are described by sensitivity variations between replicate exams). As an example, the following procedure can be used to identify stable eyes from the database: Find eyes for which there are at least three exams taken on different dates, then generate a linear fit to mean deviation (MD) vs. patient age at the time of the exam.

At 30, the present invention selects only those eyes for which the absolute value of the fit slope is less than or equal to one per year and the absolute difference between the last and the first point of the fit is less than or equal to two and one-half Additionally, those exams in a given series for which the difference between the fit to MD and the measured MD is larger than 2.5 are eliminated. Given the noise estimate procedure described below, the above method based on MD still captures large variations in sensitivities measured at any spatial eye location.

Referring again to FIG. 2, at 40 measurement samples are generated, and at 50 the measurement samples are stored. The measurement samples are defined as follows. If, for a given (stable) condition,

Sij=m,

where Sij is the eye sensitivity measured in eye exam i, and the eye spatial location is j, all eye sensitivities measured at the same location j in the other exams (i=1,2, . . . i−1,i+1, . . . , N, where N is the number of exams for the eye) are collected in sensitivity bin m. Two samples are collected for each pairwise comparison of exams. For example, if eye sensitivities k dB and m dB are measured in a given eye spatial location, value m is collected in sensitivity bin k, and value k in bin m. For the visual field, the eye sensitivity is an integer number in the range from 0 dB to about 40 dB. Thus, a separate eye sensitivity bin can be used for each observable value. Generally, all locations of the visual field test map are used except for the blind spot.

Generally, when examined, the measurement sample distributions, when normalized, represent the probability of measuring other eye sensitivity values when the “bin value” is measured at the same location. The bin value generally does not represent the mean or median value for the samples collected in that bin. Noise distribution is generally a function of the “true” eye sensitivity. However, the “true” eye sensitivity value and associated noise distribution are generally not known for any spatial location and that knowledge is not required. By pooling all eyes and locations, the above method effectively samples from a number of different “true” distributions, and assembles measurements from all possible true distributions associated with a subset of allowable eye sensitivities.

In an alternative embodiment that provides even more detailed results, the present invention may collect separate measurement samples for different subsets of spatial eye locations (e.g., upper and lower hemi-field) and, for example, for patients of different age.

In yet another embodiment, the above method may be applied to eye maps that have a larger number of measurement points (i.e., pixels) and larger dynamic range of the eye measurement. In this case it may be impractical to use separate bins for all measured values as the number of such bins could be too large for a comprehensive treatment. For those maps, several of the following modifications of the above method may be employed: First, not all locations of the eye map are sampled. The sampled locations may be predefined or selected randomly, either from the entire map or from predefined segments of the map. Second, the map may be locally averaged to produce a lower resolution map with pixels that are larger than the original pixels, and with a reduced number of pixels. Third, the value (intensity) bins (equivalent to eye sensitivity bins for the visual field test) are defined as value ranges. Then, the measurement samples are defined as the difference between the values measured by different exams. For example, if Sij is the intensity measured in exam i and spatial location j, and Sᵢⱼ∈[Sₙ, Sₙ₊₁], the values collected in bin n are Sᵢⱼ−Sₖⱼ, where k enumerates all values measured at the same location j in the other exams (k=1,2, . . . i−1,i+1, . . . , N, where N is the number of exams for the given condition).

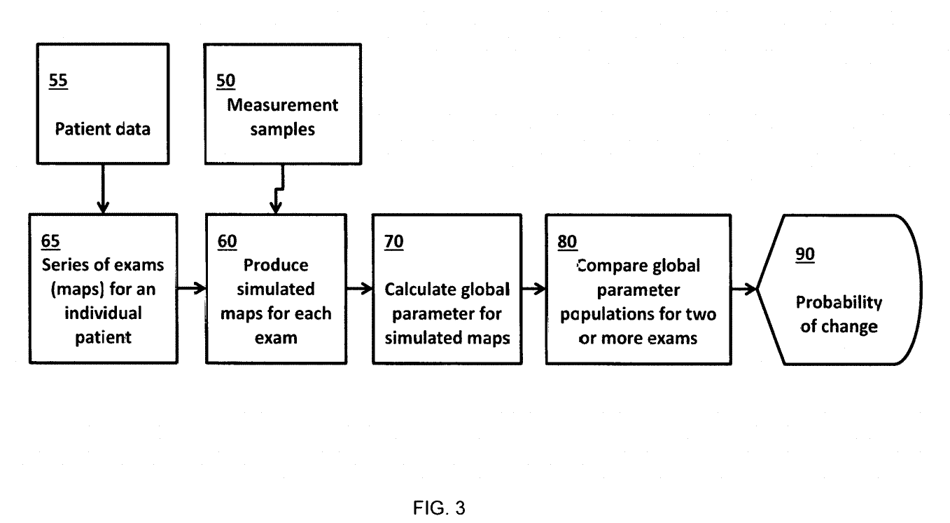

Referring now to FIG. 3, at 60 simulated maps are generated. Using the measurement samples 50, the present invention generates sets of “equivalent” maps by sampling the measurement distributions in the measurement samples 50. The equivalent map can be thought of as a map drawn from the distribution of all possible maps that includes the actually measured map. To produce an equivalent map, for each eye spatial location, the present invention substitutes the measured value (measured in dB) with a value randomly drawn, from a “X dB” sensitivity bin. That is, if visual field sensitivity values equals X dB at a location, the equivalent readings at this location are obtained through a random sampling of distributions for bin X. For the alternative embodiments described above where measurement samples are collected as differences between replicate tests (or, between tests for a stable condition) the equivalent maps are produced by substituting the measured value by X plus the measurement sample drawn from the bin containing the X value.

At 70, once the simulated maps are produced, a global parameter, or global index can be generated. As discussed above, there are several global parameters, or global indices, for example, the mean deviation (MD), pattern standard deviation (PSD), and the visual field index (VFI), among others. Now, let us define function A(I), where I is the array of values forming the map (for the eye visual field tests these are the measured retinal sensitivities). The value of A changes between normal and disease condition and thus, A may be potentially used as a measure of disease progression. The mean deviation (MD) is an example of function A used in the context of glaucoma, but there are a number of other functions (or, global indices).

The present invention enables an objective assessment of accuracy with which the value of A can be determined for any particular eye map and thus, to assign significance to the changes observed in consecutive eye examinations. In other words, the present invention can distinguish, at a given level of significance, between the variation, due solely to the measurement noise, and actual changes in the eye visual field.

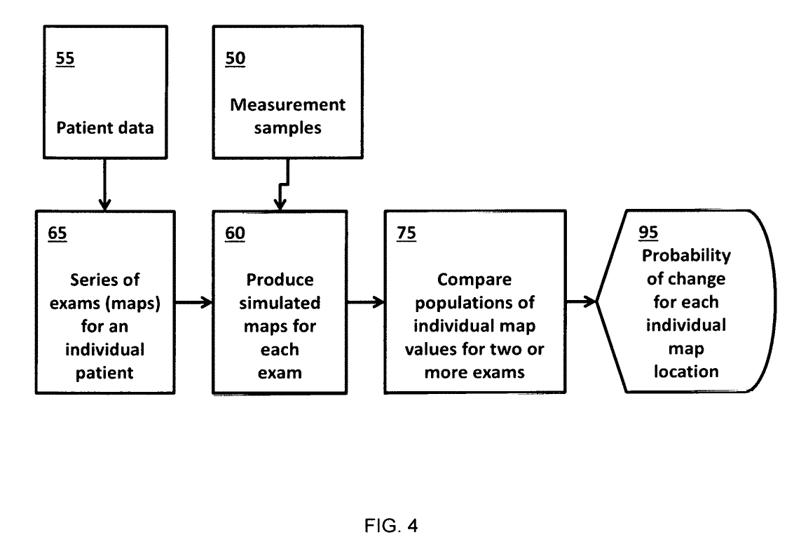

Referring now to FIG. 4, changes at individual map locations may be assessed in a similar manner as described above in connection with FIG. 3. Here, the simulated maps are used to analyze progression separately for all individual map locations, using the populations of simulated local sensitivities, and probability of progression is determined for each map location. For example, once the simulated maps are produced at 60, simulated maps are used to analyze progression separately for all individual map locations at 75, using the populations of simulated local sensitivities. Then, at 95 the probability of change, or progression is determined for each map location.

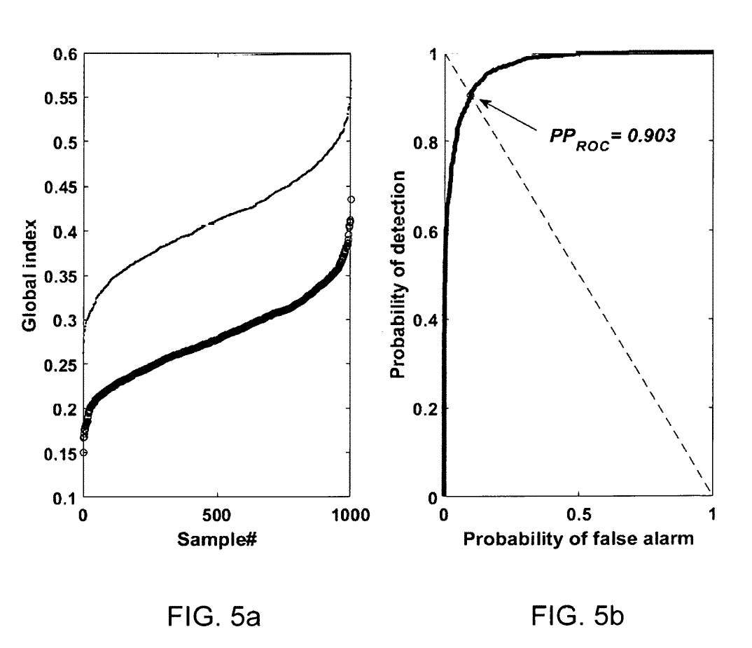

FIGS. 5a-b illustrate one method of the present invention. For a given test map, a large number (say, one thousand) of “equivalent” fields is produced by random resampling of the empirically determined measurement distributions, as discussed above at 60. The function A is calculated for all simulated fields, for two maps being compared as shown in FIG. 5a, which shows the values of A for the first map (lower line, comprised of circles) and values of A for the second map (the upper line, comprised of dots). Then, the receiver operating characteristic (ROC) probability of progression, PP_{ROC}, is determined using A values obtained for the sets of equivalent maps for two eye exams being compared, see FIG. 5b. The range of PP_{ROC} is from 0 to 1. Null value is obtained for regressing maps, PP_{ROC}=0.5 for maps that are identical. Alternatively, the present invention can determine a confidence interval for A: [A_{L}, A_{H}]. For example, for a 95% confidence, A_{L}, and A_{H} are the low and high values of the range containing 95% of calculated values (with 2.5% of values being smaller than A_{L}, and 2.5% being larger than A_{H}). For two eye exams for a given patient, the present invention can determine a map progression (or, regression), with relevant confidence, if the A confidence ranges for two eye exams being compared do not overlap.

Referring to FIG. 5b, the ROC curve is a plot of the probability of detection (PD or, sensitivity) versus the probability of a false alarm, or mis-diagnosis (PFA or, 1-specificity). PP_{ROC}, equal to 0.903 in this example, is PD calculated at the intersection of the ROC curve with the plot diagonal (dashed line). The corresponding probability of regression is equal to 1−PP_{ROC}. The receiver operating characteristic (ROC), or ROC curve, describes performance of a binary classifier (e.g., progression vs. no progression). It is a plot of true positive rate (probability of detection, or sensitivity) versus false positive rate (probability of false alarm or, 1-specificity) at various detection thresholds. The ROC is a non-parametric method as the curve may be produced without any assumptions regarding the data distributions. Other tests may be employed to compare the simulated distributions generated by the present invention. For example, these tests may include the Mann-Whitney (Wilcoxon rank-sum) test, the Kolmogorov-Smirnov test, the Student's t-test, analysis of variance (ANOVA) tests, and others.

Referring back to FIG. 3, with patient data 55 from two eye exams that generate eye maps 65, the present invention can determine a probability of map progression (or, regression). That is, at 80 values for a global index are generated for the simulated eye examination maps as described above, and at 90, a measure of the probability of disease progression is determined. Similar steps are performed in FIG. 4, where a probability of disease progression is determined for individual map locations.

The present invention enables the quantitative assessment of progression based on two consecutive eye tests. However, when a time series of tests is available, the use of a polynomial fit to the data increases the sensitivity of progression detection. As for pairwise comparisons, each measured eye map in the series is used to produce a large number (e.g., 1000) of equivalent fields and corresponding populations of the global index (or, indices). In this embodiment, in order to estimate the fit uncertainty, the present invention uses another level of resampling. For every time point in the series (for example, patient age), the present invention randomly selects one value from the (simulated) global index population for that age and produces a polynomial fit (linear, quadratic or higher order, depending on the data characteristics) to that resampled series (global index vs. time). Typically, 500 to 1000 such fits are produced. The confidence interval for the fit is determined as the interval containing 95% of fitted values for each data point. The procedure imposes limits on possible values of the first and last point in a series—all that matters for progression evaluation is the change between those points. The progression probability is determined through comparison (for example, the ROC analysis described above) of the distributions of fitted values between the first and the last test in the series.

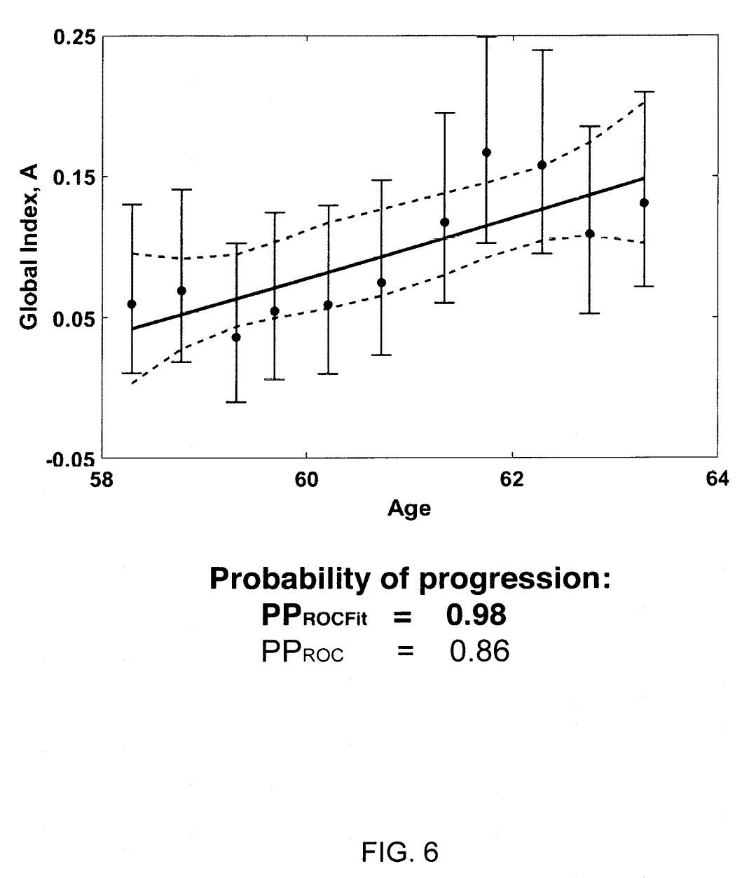

Referring now to FIG. 6, an example of the results produced by the present invention is illustrated. In this example, the map resampling method described above and a global parameter, or global index are shown. The value of global index A (in this example, a perimetric progression index, described below) is plotted versus the patient's age at the time of examination. The error bars show 95% confidence level evaluated for each exam in the series. The solid line is a quadratic polynomial fit to the measured points, with the dashed lines showing the 95% confidence interval for the fit. PP_{ROC} is the ROC probability of progression between the first and the last eye exam in the series obtained through direct comparison of the two. PP_{ROCFit} is the probability of progression between the first and last eye exam obtained for the quadratic fit, as described above. Generally, PP_{ROCFit} is used as a preferred measure of progression probability. This series of test results indicates a high probability of disease progression. Typically, the progression would be indicated for a probability of progression above 0.95, which corresponds to a probability of false alarm (or mis-diagnosis) of about 0.05.

The feature of the present invention is that it may be used to directly analyze the significance of observed variations in local values (i.e., individual features of the eye map or, map pixels). By resampling the measurement sample distributions, as described above, the present invention simulates populations of possible measurements for individual locations. Comparison of these populations obtained for two time points, using either the ROC approach (illustrated above) or any other suitable approach, permits the determination of the probability of progression/regression of disease.

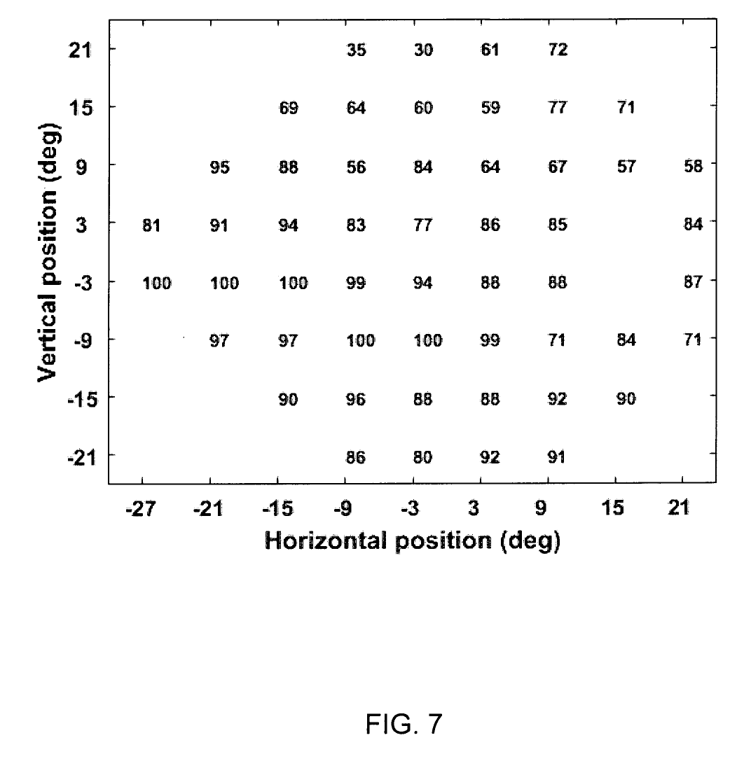

Referring now to FIG. 7, a diagnostic map generated according to the present invention is illustrated. The map shows an example of the result of pointwise analysis. The probability of progression (in %) is displayed at each map location. The black areas corresponding to 0% (probability of progression, or disease equal to 0, and probability of regression equal to 1) and white corresponding to 100% (probability of progression, or disease equal to 1). With this clear presentation of the results generated by the present invention, a doctor can quickly determine the results of a patient's eye exam with confidence.

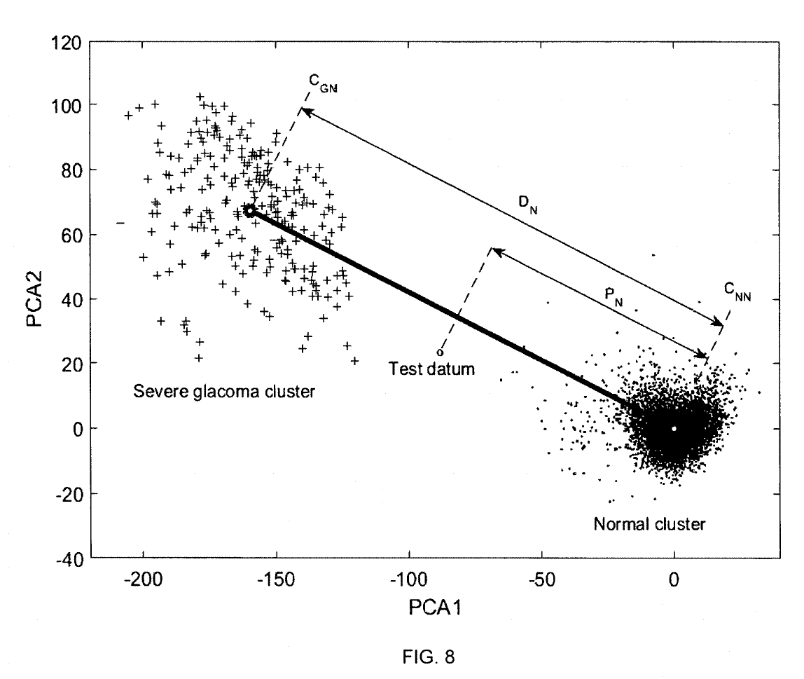

Referring now to FIG. 8, another embodiment of the present invention is illustrated. This embodiment is the Perimetric Progression Index (PPI), which may be considered a new global index. The PPI roughly represents the geometric distance on a trajectory between the clusters of normal (no perimetric glaucoma) and severe glaucoma fields. These clusters represent extreme states in the space of all perimetric measurements and it may be surmised that all eye visual field measurements are located between them (or, on the trajectory between them). Importantly, the normal and severe glaucoma fields are relatively easy to identify. The PPI is calculated using principal component analysis (PCA) and its nominal range is from 0 (normal field) to 1 (severe glaucoma field), although values outside that range are allowed. The PCA analysis is performed not on the eye sensitivity values but using a set of summary parameters that describe the eye visual field. The summary parameters may include: 1) Histograms. Sensitivity histograms are derived for the entire eye map and the following sub-regions: inferior, superior, inferior nasal, superior nasal, superior temporal, inferior temporal, superior paracentral, and inferior paracentral. Histograms, absolute differences between inferior and superior regions, and region averaged sensitivities are considered. 2) Averages. Mean value, standard deviation, maximum, minimum and median values are calculated for the entire visual field, superior and inferior halves, and for quarters of the visual field (nasal superior, temporal superior, etc.). Additionally, the regional values are sorted by the relevant mean values. 3) Defect curve. The visual field data are presented as a series with monotonically decreasing values. In the present invention, the curve is normalized to the mean of the eight largest eye sensitivities. 4) Maximum gradient. For each eye measurement point, the maximum eye sensitivity difference between the measured sensitivity and the sensitivity measured in adjacent eye points is calculated. A histogram of the maximum gradient is used.

As mentioned above, part of the PPI embodiment uses principal component analysis (PCA), which is a statistical procedure that uses an orthogonal transformation to convert a set of observations of possibly correlated variables into a set of values of linearly uncorrelated variables called principal components. The number of principal components is less than or equal to the number of original variables. This transformation is defined in such a way that the first principal component has the largest possible variance (that is, accounts for as much of the variability in the data as possible), and each succeeding component in turn has the highest variance possible under the constraint that it is orthogonal to the preceding components. The resulting vectors are an uncorrelated orthogonal basis set. The principal components are orthogonal because they are the eigenvectors of the covariance matrix, which is symmetric. PCA is sensitive to the relative scaling of the original variables.

Referring again to FIG. 8, the Perimetric Progression Index (PPI) is found using the following procedure: A principal component model is generated by assembling a collection of eye visual field maps for normal (disease free) patients. Then, a collection of eye visual field maps for patients with severe glaucoma is assembled. Then, the principal components for the normal cyc maps summary parameters are determined using principal component analysis (PCA), described above. Then, one to ten of the most significant principal components (i.e., the “normal model principal components”) are used to determine C_{NN}, where C_{NN} is the position of the geometrical center of the normal cluster in the principal component space for the normal eye maps. Then, the normal model principal components are used to determine C_{GN}, where C_{GN} is the position of the geometrical center of the severe glaucoma cluster in the principal component space for normal maps. Then, D_{N} is determined, which is the geometric distance between C_{NN} and C_{GN}. These elements are illustrated in FIG. 8. In this example, the two most significant PCA components are used (PCA1 and PCA2).

Also, the principal components are determined for the severe glaucoma maps summary parameters using principal component analysis (PCA), as described above. Then one to ten of the most significant principal components (i.e., the “glaucoma model principal components”) are used to determine C_{GG}, where C_{GG} is the position of the geometrical center of the severe glaucoma cluster in the principal component space for the severe glaucoma maps. Then, the glaucoma model principal components are used to determine C_{NG}, where C_{NG} is the position of the geometrical center of the normal cluster in the principal component space for the severe glaucoma maps. And, D_{G} is determined, which is the geometric distance between C_{GG} and C_{NG}.

Referring again to FIG. 8, the PPI for a single eye examination is determined as follows. In this example, the summary parameters for the eye examination map are determined. The normal model principal components are used to determine the position of the eye map (test datum) in the principal component space for normal maps. The normal model trajectory is defined as a straight line connecting C_{NN} and C_{GN}, which are the centers of normal and severe glaucoma clusters in the principal component space for normal maps. P_{N} is then determined, which is the geometrical distance between C_{NN} and the position of eye datum, determined on the normal model trajectory. PPI_{N}=P_{N}/D_{N} . This is the distance between the position of a given eye map in the principal component space for normal eye maps, normalized to the distance between the centers of normal and severe glaucoma clusters in the principal component space for normal eye maps.

In another example, the glaucoma model trajectory is defined as a straight line connecting C_{NG} and C_{SG}, which are the centers of the normal and the severe glaucoma clusters in the principal component space for severe glaucoma maps. P_{G} is calculated as the geometrical distance between C_{NG} and the position of eye datum, determined on the glaucoma model trajectory. PPI_{G}=P_{G}/D_{G}. This is the distance between the position of a given eye map in the principal component space for severe glaucoma maps, normalized to the distance between the centers of normal and severe glaucoma clusters in the principal component space for severe glaucoma maps.

At that point, PPI is either of: PPI_{N}, PPI_{G} or a combination of the two, based on the data from the single eye examination.

One feature of PPI is that it correlates very closely with the mean deviation (MD) and the visual field index (VFI) global indices, with the Pearson correlation coefficients typically less than −0.95. However, the slope of PPI dependence on MD and VFI is somewhat steeper than would be expected for a perfectly linear relationship, with the PPI “saturating” for low values of MD and VFI. That is, PPI reacts faster to the eye visual field changes in the vicinity of normal fields. This enables for an earlier determination of the onset of glaucoma with a higher confidence. The use of PPI in the progression method embodiment of this invention (described above) results is a “self-consistent” method that has false alarm rate approximately equal to (1-probability of progression).

As discussed above present invention is a self-consistent system and method that produces a comprehensive model of measurement noise. It also enables quantitative assessment of the probability of change in a global index (progression/regression) and, by extension, disease progression. In contrast to other approaches, a statistically significant change in the parameter value (that is, a change that cannot be attributed to intrinsic, random fluctuations and thus, is mostly due to the change in patient condition) can be determined by comparison of only two diagnostic results. Acquisition and analysis of a larger number of diagnostic results (time histories) helps in progression determination but is not necessary.

The present invention can also be applied to other fields, and to other types of diagnostic tests that include measurement noise. For example, changes in the eye structure may be observed with a number of modern instruments. In the context of glaucoma diagnosis, of particular interest are the measurements of the retinal nerve fiber layer (RNFL) thickness and its changes. Such measurements are performed with instruments using scanning laser polarimetry and optical coherence tomography (OCT).

High-speed Fourier-domain optical coherence tomography (FD-OCT) can be used to map ganglion cell complex (GCC) thickness. Several glaucoma diagnostic parameters based on the GCC map are produced including: the overall average thickness and the difference between superior and inferior hemispheric averages (GCC-SID). The maps of GCC loss are also computed as the fractional deviation (FD) map and the pattern deviation (PD) map. The FD map is the GCC map minus the normal reference map divided by the normal reference map. The pattern map is the GCC thickness map normalized (divided) by its own overall average. The PD map is the pattern map under consideration minus the normal reference pattern. The FD map shows the percentage of GCC loss. The PD map shows how the GCC pattern differs from normal. Three pattern-based diagnostic parameters (global indices) are then computed from the 2 derivative maps. The focal loss volume (FLV) is the sum FD in the region where there is significant focal loss. Global loss volume (GLV) is the sum of FD in areas where FD is negative. Pattern coefficient of variation (PCV) is the root mean square of the PD map.

The present invention can be applied to the above maps obtained from the laser polarimetry, optical coherence tomograpgy (OCT), and Fourier-domain optical coherence tomography (FD-OCT) methods. As discussed above, the present invention uses maps generated by a Humphrey Visual Field Analyzer. Then, the present invention applies a novel analysis to the maps, and data obtained therefrom, to provide a novel output that assists doctors in determining disease progression. The same methods and systems can be applied to the maps and data generated by instruments measuring the structure of RNFL and GCC.

As will be appreciated by one of skill in the art, embodiments of the present invention may be provided as methods, systems, or computer program products. Accordingly, the present invention may take the form of an entirely hardware embodiment, an entirely software embodiment or an embodiment combining software and hardware aspects. Furthermore, the present invention may take the form of a computer program product, or a non-transitory machine readable storage medium which is embodied on one or more computer-usable storage media (including, but not limited to, disk storage, CD-ROM, optical storage, and so forth) having computer-usable program code embodied therein.

The present invention has been described with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, embedded processor or other programmable data processing apparatus to produce a machine, such that the instructions, which execute via the processor of the computer or other programmable data processing apparatus, create means for implementing the functions specified in the flowchart and/or block diagram block or blocks.

These computer program instructions may also be stored in a computer-readable memory that can direct a computer or other programmable data processing apparatus to function in a particular manner, such that the instructions stored in the computer-readable memory produce an article of manufacture including instruction means which implement the function specified in the flowchart and/or block diagram block or blocks.

The computer program instructions may also be loaded onto a computer or other programmable data processing apparatus to cause a series of operational steps to be performed on the computer or other programmable apparatus to produce a computer implemented process such that the instructions which execute on the computer or other programmable apparatus provide steps for implementing the functions specified in the flowchart and/or block diagram block or blocks.

Further, although process steps, method steps, algorithms or the like may be described in a sequential order, such processes, methods and algorithms may be configured to work in alternate orders. In other words, any sequence or order of steps that may be described does not necessarily indicate a requirement that the steps be performed in that order. The steps of processes described herein may be performed in any order practical. Further, some steps may be performed simultaneously.

The term “computer-readable medium” or “non-transitory machine readable storage medium” as used herein refers to any medium that participates in providing data (e.g., instructions) which may be read by a computer, a processor or a like device. Such a medium may take many forms, including but not limited to, non-volatile media, volatile media, and transmission media. Non-volatile media include, for example, optical or magnetic disks and other persistent memory. Volatile media include dynamic random access memory (DRAM), which typically constitutes the main memory. Transmission media include coaxial cables, copper wire and fiber optics, including the wires that comprise a system bus coupled to the processor. Transmission media may include or convey acoustic waves, light waves and electromagnetic emissions, such as those generated during radio frequency (RF) and infrared (IR) data communications. Common forms of computer-readable media include, for example, a floppy disk, a flexible disk, hard disk, magnetic tape, any other magnetic medium, a CD-ROM, DVD, any other optical medium, punch cards, paper tape, any other physical medium with patterns of holes, a RAM, a PROM, an EPROM, a FLASH-EEPROM, any other memory chip or cartridge, a carrier wave as described hereinafter, or any other medium from which a computer can read.

Various forms of computer readable media may be involved in carrying sequences of instructions to a processor. For example, sequences of instruction (i) may be delivered from RAM to a processor, (ii) may be carried over a wireless transmission medium, and/or (iii) may be formatted according to numerous formats, standards or protocols, such as Bluetooth, TDMA, CDMA, and 3G.

It is to be noticed that the term “comprising”, used in the claims, should not be interpreted as being limitative to the means listed thereafter. Thus, the scope of the expression “a device comprising means A and B” should not be limited to devices consisting only of components A and B. It means that with respect to the present invention, the only relevant components of the device are A and B. The terms “an embodiment”, “embodiment”, “embodiments”, “the embodiment”, “the embodiments”, “one or more embodiments”, “some embodiments”, and “one embodiment” mean “one or more (but not all) embodiments of the present invention(s)” unless expressly specified otherwise. The terms “including”, “comprising” and variations thereof mean “including but not limited to”, unless expressly specified otherwise. The enumerated listing of items does not imply that any or all of the items are mutually exclusive, unless expressly specified otherwise. The terms “a”, “an” and “the” mean “one or more”, unless expressly specified otherwise.

Thus, it is seen that a visual field progression system (VFPS) is provided. One skilled in the ail will appreciate that the present invention can be practiced by other than the above-described embodiments, which are presented in this description for purposes of illustration and not of limitation. The specification and drawings are not intended to limit the exclusionary scope of this patent document. It is noted that various equivalents for the particular embodiments discussed in this description may practice the invention as well. That is, while the present invention has been described in conjunction with specific embodiments, it is evident that many alternatives, modifications, permutations and variations will become apparent to those of ordinary skill in the art in light of the foregoing description. Accordingly, it is intended that the present invention embrace all such alternatives, modifications and variations as fall within the scope of the appended claims. The fact that a product, process or method exhibits differences from one or more of the above-described exemplary embodiments does not mean that the product or process is outside the scope (literal scope and/or other legally-recognized scope) of the following claims.

What is claimed is:

1. A method of identifying a change in an eye to detect a probability of a disease progression, the method comprising the steps of:
providing a database comprising a multiplicity of eye examination maps obtained from a plurality of different eyes, with each eye examination map comprising visual field data;
selecting maps of eyes from the database that show substantially no change between their examinations;
generating measurement data from the selected eye maps by collecting a plurality of measurements made at a plurality of visual field locations from each selected eye map;
generating a plurality of simulated eye examination maps by randomly sampling a value from the measurement data for each visual field location; and
employing a global index to generate an output using each of the plurality of simulated eye examination maps, where the output provides a measure of the probability of the disease progression.

2. The method of claim 1, where each of the plurality of simulated maps is generated by substituting the plurality of measurements made at each of the plurality of visual field locations from each selected eye map with a value randomly drawn from the plurality of measurements.

3. The method of claim 1, where the global index is selected from a group consisting of: a mean deviation, a pattern standard deviation, a visual field index, a perimetric progression index, and a combination of two or more thereof.

4. The method of claim 1, further comprising the step of:
employing a statistical method to generate a numerical quantity that indicates a probability of the disease progression using the global index for each of the plurality of simulated eye examination maps; and
generating a disease progression probability map, the map comprising the plurality of visual field locations, with each visual field location displaying the probability of the disease progression.

5. The method of claim 4, where the statistical method is selected from the group consisting of: a receiver operating characteristic (ROC) method, a Mann-Whitney method, a Kolmogorov-Smirnov method, a Student's t-test, an analysis of variance, and combination of two or more thereof.

6. The method of claim 1, where each of the multiplicity of eye examination maps comprises a retinal sensitivity map obtained from an ophthalmic perimeter device.

7. The method of claim 1, where the visual field data of each eye examination map comprises a plurality of numbers that indicate a patient's threshold visual eye sensitivity in decibel (dB) units.

8. A non-transitory machine readable storage medium having stored thereon a computer program for detecting a probability of a disease progression, the computer program comprising a routine of set instructions for causing the machine to perform the steps of:
accessing a database comprising a multiplicity of eye examination maps obtained from a plurality of different eyes, with each eye examination map comprising visual field data;
selecting maps of eyes from the database that show no change between their examinations;
generating measurement data from the selected eye maps by collecting a plurality of measurements made at a plurality of visual field locations from each selected eye map;
generating a plurality of simulated eye examination maps by randomly sampling a value from the measurement data for each visual field location; and
employing a global index to generate an output using each of the plurality of simulated eye examination maps, where the output provides a measure of the probability of the disease progression.

9. The non-transitory machine readable storage medium of claim 8, where each of the plurality of simulated maps is generated by substituting the plurality of measurements made at each of the plurality of visual field locations from each selected eye map with a value randomly drawn from the plurality of measurements.

10. The non-transitory machine readable storage medium of claim 8, where the global index is selected from a group consisting of: a mean deviation, a pattern standard deviation, a visual field index, a perimetric progression index, and a combination of two or more thereof.

11. The non-transitory machine readable storage medium claim 8, further comprising the step of:
employing a statistical method to generate an output using each of the plurality of simulated eye examination maps; and
generating a disease progression probability map, the map comprising the plurality of visual field locations, with each visual field location displaying the probability of the disease progression.

12. The non-transitory machine readable storage medium of claim 8, where the statistical method is selected from the group consisting of: a receiver operating characteristic (ROC) method, a Mann-Whitney method, a Kolmogorov-Smirnov method, a Student's t-test, an analysis of variance, and combination of two or more thereof.

13. The non-transitory machine readable storage medium of claim 8, where each of the multiplicity of eye examination maps comprises a retinal sensitivity map obtained from an ophthalmic perimeter device.

14. The non-transitory machine readable storage medium of claim 8, where the visual field data of each eye examination map comprises a plurality of numbers that indicate a patient's threshold visual eye sensitivity in decibel (dB) units.

15. A method of identifying a change in an eye to detect a probability of a disease progression, the method comprising the steps of:
providing a first database comprising a multiplicity of eye examination maps obtained from a plurality of different eyes exhibiting no signs of a disease, with each eye examination map comprising visual field data;
providing a second database comprising a multiplicity of eye examination maps obtained from a plurality of different eyes exhibiting signs of the disease, with each eye examination map comprising visual field data;
generating a plurality of summary parameters for the eye examination maps in both databases;
deriving a plurality of principal components by a principal component analysis, for the plurality of summary parameters representing eye examination maps contained in the first database;
using at least one most significant principal component from the plurality of principal components derived from the first database, to determine geometrical centers of the data contained in the first and second databases;
defining a first trajectory as a line connecting the geometrical centers of the data represented by the principal components contained in the first and second databases;
deriving a plurality of principal components by a principal component analysis, for the plurality of summary parameters representing examination maps contained in the second database;
using at least one most significant principal component, from the plurality of principal components derived for the second database, to determine geometrical centers of the data contained in the first and second databases;
defining a second trajectory as a line connecting the geometrical centers of the data represented by the principal components contained in the first and second databases; and
determining the disease progression by a location of a single eye test datum on the first trajectory, the second trajectory, or a combination of the two.

16. The method of claim 15, where each of the multiplicity of eye examination maps comprises a retinal sensitivity map obtained from an ophthalmic perimeter device.

17. The method of claim 15, where the visual field data of each eye examination map comprises a plurality of numbers that indicate a patient's threshold visual eye sensitivity in decibel (dB) units.

18. The method of claim 15, where the first and second summary parameters are selected from a group consisting of: an eye sensitivity histogram, an eye sensitivity average, an eye sensitivity defect curve, a histogram of a maximum gradient that is generated by finding a maximum eye sensitivity difference between a measured eye sensitivity and an eye sensitivity measured in an adjacent visual field point, and a combination of two or more thereof.

19. The method of claim 15, where the summary parameters are selected from a group consisting of: an age of a patient, a median deviation, a visual field index, a pattern standard deviation, and a combination of two or more thereof.
